(19) United States
(12) Patent Application Publication
(10) Pub. No.: US 2008/0045988 A1
(43) Pub. Date: Feb. 21, 2008

(54) Tongue deodorizing device

(76) Inventors: Dominic Abbott, Pittsburgh, PA (US); Chun Lim Abbott, Pittsburgh, PA (US)

Correspondence Address: Epstein & Gerken, Suite 340, 1901 Research Boulevard, Rockville, MD, 20850, US

(21) Appl. No.: 11/507,077

(22) Filed: Aug. 18, 2006

(51) Int. Cl. A61B 17/24

(52) U.S. Cl. 606/161

(57) ABSTRACT

A tongue deodorizing device comprises a tongue contacting instrument having a stainless steel tissue rubbing surface for being rubbed against the tongue and having a water supply for supplying water to a fluid transport channel of the tongue contacting instrument for discharge adjacent the tongue rubbing surface. The tongue rubbing surface in contact with the tissue of the tongue reacts with the water to deodorize the tongue via a non-mechanical reaction. The tongue contacting instrument may also comprise a tongue scraping element for mechanically scraping the tongue to reduce odor via mechanical removal of odor-causing material from the tongue.

### BACKGROUND OF THE INVENTION

1. Field of the Invention

The present invention relates generally to tongue deodorizing devices and, more particularly, to tongue deodorizing devices that cause a non-mechanical reaction to occur which reduces odor arising from the tongue.

2. Brief Discussion of the Related Art

Halitosis, otherwise known as bad breath, is a troublesome condition that afflicts many individuals despite regular brushing and flossing of the teeth and the use of commercial mouthwash. Halitosis oftentimes presents professional and personal obstacles for the afflicted individuals including embarrassment, shyness, social difficulties, low self-esteem, anxiety and lack of confidence. Although halitosis can be caused by numerous etiological factors, the vast majority of cases originate in the mouth and predominantly the tongue. Volatile sulfur compounds (VSC), including hydrogen sulfide, methyl mercaptan and dimethyl sulfide, generated in the mouth and especially from the tongue are the primary cause of halitosis. Volatile sulfur compounds result from the proteolytic degradation of various sulfur-containing substances, such as food debris, saliva, blood and epithelial cells, by predominantly anaerobic gram-negative oral microorganisms. Most of the microorganisms that contribute to the development of volatile sulfur compounds are considered periodontal pathogens.

The tongue, and especially the dorsum or top surface of the tongue, presents a large surface area conducive to the accumulation of oral debris, microorganisms and other substances associated with the production of volatile sulfur compounds. The dorsum is characterized by an irregular or rough surface topography that promotes adhesion thereto of oral debris, microorganisms and other substances associated with the production of volatile sulfur compounds. In particular, the dorsum exhibits many cryptolymphatic units, papillary structures, fissures and crevices that harbor large quantities of the oral debris, microorganisms and other substances. Consequently, a coating normally accumulates on the dorsum of the tongue and, in many individuals, the coating accumulates to a considerable thickness. The coating normally includes food residue, microorganisms including bacteria and fungus, deposits of desquamated epithelial cells, and leukocytes. The coating provides an environment very favorable to the production of volatile sulfur compounds. In particular, the coating provides an oxygen deprived environment that favors the proliferation of proteolytic anaerobic flora and a bacterial biofilm involved in the development of volatile sulfur compounds. The coating or material on the surface of the tongue tends to be tenaciously adhered to the tongue. Ordinary toothbrushes have not been found to be effective in reducing tongue coating to a significant extent when used to brush the surface of the tongue.

Accumulation of a coating or material on the tongue is exacerbated in many individuals due to low saliva production. The secretion of saliva, which is controlled by the autonomic nervous system, has the beneficial effect of eliciting a swallowing reaction that assists in removing bacteria, food debris and other substances from the mouth. In addition, saliva has a diluting and/or buffering effect on bacteria and bacterial waste products implicated in halitosis. Where saliva production is low, the benefits derived from the presence of saliva are diminished, and conditions are created that encourage the proliferation of odor-causing organisms and substances as well as their accumulation on the tongue. Low saliva production is found in a large percentage of the population and may be chronic, i.e. a condition known as “dry mouth” or xerostomia, or may be episodic. Factors that may cause or contribute to low saliva production include medicinal side effects, systemic disease, pathologic changes in the salivary glands, prolonged speaking, mouth breathing, fasting, stress, and old age. Even where saliva production is normal, the unstimulated saliva flow rate is only about 0.2 to 0.4 ml per minute. The small quantity of saliva obtained from normal saliva production limits the extent to which saliva can deter the proliferation and accumulation of odor-causing organisms and substances. Accordingly, it is still very common for halitosis to occur in the absence of salivary deficiencies.

Tongue scrapers have been proposed for scraping the surface of the tongue to mechanically remove some of the coating therefrom. Tongue scrapers attempt to alleviate halitosis indirectly, through the use of a mechanical scraping action to reduce the quantity of odor-producing material present on the surface of the tongue. Tongue scrapers in which the scraping element may be made of stainless steel have been proposed as represented by U.S. Pat. No. 3,683,924 to Louie, No. 5,061,272 to Reese, No. 5,778,475 to Garcia, No. 5,893,860 to Ripich et al, No. 5,913,346 to Narwani, No. 5,916,228 to Ripich et al, No. 5,957,942 to Yudelman, No. 6,056,763 to Parsons, No. 6,099,540 to Ho and No. 7,029,484 B2 to Ripich, and by U.S. Patent Application Publications No. US 2002/0128673 A1 to Ripich et al, No. US 2004/0134008 A1 to Pham and No. US 2006/0025791 A1 to Ripich et al. In the tongue scrapers of the aforementioned patents and patent application publications, the use of stainless steel is not necessary for operation of the tongue scrapers to mechanically remove material from the tongue. Some of the tongue scrapers include channels or recesses to collect material scraped from the tongue as represented by U.S. Pat. No. 5,061,272 to Reese, No. 5,778,475 to Garcia, No. 5,957,942 to Yudelman, No. 6,099,540 to Hoe and No. 7,029,484 B2 to Ripich, and by U.S. Patent Application Publications No. US 2002/0128673 A1 and No. US 2006/0025791 A1 to Ripich et al. U.S. Pat. No. 7,029,484 B2 to Ripich and U.S. Patent Application Publications No. US 2002/0128673 A1 and No. US 2006/0025791 A1 to Ripich et al all pertain to tongue scrapers that are connectible to a source of suction for removal of material that the tongue scraper has collected from the tongue.

U.S. Pat. No. 5,779,654 to Foley et al, No. 6,132,445 to Pavanelli and No. 6,139,558 to Wagner, and U.S. Patent Application Publication No. US 2003/0083680 A1 to Jousson relate to tongue scrapers in which the mechanical scraping action of the tongue scrapers is assisted by fluid flow to the mouth or tongue in order to flush and/or loosen material from the tongue.

U.S. Patent Application Publication No. US 2002/0107537 A1 to Singh discloses a breath freshening wand for removing specific food odors, namely, odors from poultry, garlic, onion and fish. At least in the case of onion and garlic, these odors come from allyl methyl sulfide (AMS), as opposed to hydrogen sulfide, methyl mercaptan and dimethyl sulfide. The breath freshener wand operates by rubbing a metal head of the wand against tissue surfaces within the mouth in the presence of saliva, as opposed to plain water. The metal can be, but is not required to be, stainless steel. In contrast to plain water, saliva contains not only water but also mucous, enzymes and electrolytes including sodium and potassium. Furthermore, large quantities of bicarbonate ions are secreted in saliva. Accordingly, saliva and plain water are different compounds having different compositional and reactive properties. In addition, the amount of water normally present in the mouth is relatively miniscule given the low flow rate at which saliva is normally produced and the fact that water is but one component of saliva.

### SUMMARY OF THE INVENTION

The present invention is generally characterized in a tongue deodorizing device comprising a tongue contacting instrument and a water supply connectible to the tongue contacting instrument. The tongue contacting instrument includes a tongue contacting member and a handle attached to the tongue contacting member. The tongue contacting member has a tongue contacting side including a tongue rubbing surface for being rubbed against the tongue. The tongue rubbing surface extends longitudinally between forward and rearward ends of the tongue contacting member and extends laterally along the tongue contacting member. The tongue rubbing surface is a smooth, solid surface of stainless steel reactive with water to reduce odor from the tongue via a non-mechanical reaction. Preferably, the tongue rubbing surface has a convex curvature in the longitudinal direction and a convex curvature in the lateral direction. The handle has a forward end connected to the rearward end of the tongue contacting member and has a rearward end connectible to the water supply. A fluid transport channel extends within the handle and has a fluid inlet at the rearward end of the handle for receiving water from the Water supply and has a fluid outlet adjacent the tongue rubbing surface at, near or adjacent the rearward end of the tongue contacting member for discharging water from the fluid transport channel to flow along the tongue rubbing surface. The water supply comprises a container for holding a quantity of water, a conduit having ends connectible with the container and with the rearward end of the handle of the tongue contacting instrument to establish fluid communication between the container and the fluid inlet, and a flow control mechanism for controlling the flow of water from the container to the fluid inlet. The container includes a discharge orifice for fluid communication with the conduit and through which water is discharged from the container by gravitational force. The container can include a handle by which the container may be hung or suspended from an available structure to assist gravitational discharge of water through the discharge orifice. The conduit includes a length of flexible tubing having a lumen extending entirely therethrough. The flow control mechanism includes a clamp attachable to the conduit for selectively opening and closing the lumen of the conduit to control the flow of water therethrough. In a closed position for the clamp, the clamp presses against the conduit and collapses the conduit wall to constrict or close the lumen. In an open position for the clamp, the lumen of the conduit is not constricted. The tongue deodorizing device is preferably designed so that a quantity of or about 6-8 ounces of water in the container is discharged from the fluid outlet in about 20-30 seconds.

The tongue contacting member preferably has a second tongue contacting side including a tongue scraping element for scraping the tongue to reduce odor from the tongue via a mechanical action, thereby providing two different modes of tongue deodorization. The second tongue contacting side comprises the tongue scraping element and a debris collecting surface bordered by the tongue scraping element for collecting material or debris scraped from the tongue by the tongue scraping element. The tongue scraping element includes a tongue scraping surface connecting the tongue rubbing surface to the debris collecting surface, and a tongue scraping edge formed by the juncture of the tongue scraping surface with the debris collecting surface. The tongue scraping element extends along a perimeter of the tongue contacting member and preferably follows a continuously curving path between lateral sides of the rearward end of the tongue contacting member. The tongue scraping element may follow an elliptical, ovoid or oblong path between the lateral sides of the rearward end of the tongue contacting member. Preferably, the tongue scraping edge is disposed in a lateral plane that is offset from a central longitudinal axis of the handle in parallel relation. The debris collecting surface preferably has a concave curvature in the longitudinal direction and a concave curvature in the lateral direction. The debris collecting surface defines a debris collecting cavity bordered by the tongue scraping edge.

Various objects, benefits and advantages of the claimed invention will become apparent from the following description of a preferred embodiment taken in conjunction with the accompanying drawings.

### BRIEF DESCRIPTION OF THE DRAWINGS

FIG. 1 is an exploded perspective view of a tongue deodorizing device according to the present invention.

FIG. 2 is a plan view of a tongue rubbing side of a tongue contacting instrument of the tongue deodorizing device.

FIG. 3 is a longitudinal side view of the tongue contacting instrument.

FIG. 4 is a plan view of a tongue scraping side of the tongue contacting instrument.

FIG. 5 is a distal end view of the tongue contacting instrument.

FIG. 6 is a proximal end view of the tongue contacting instrument.

### DESCRIPTION OF A PREFERRED EMBODIMENT

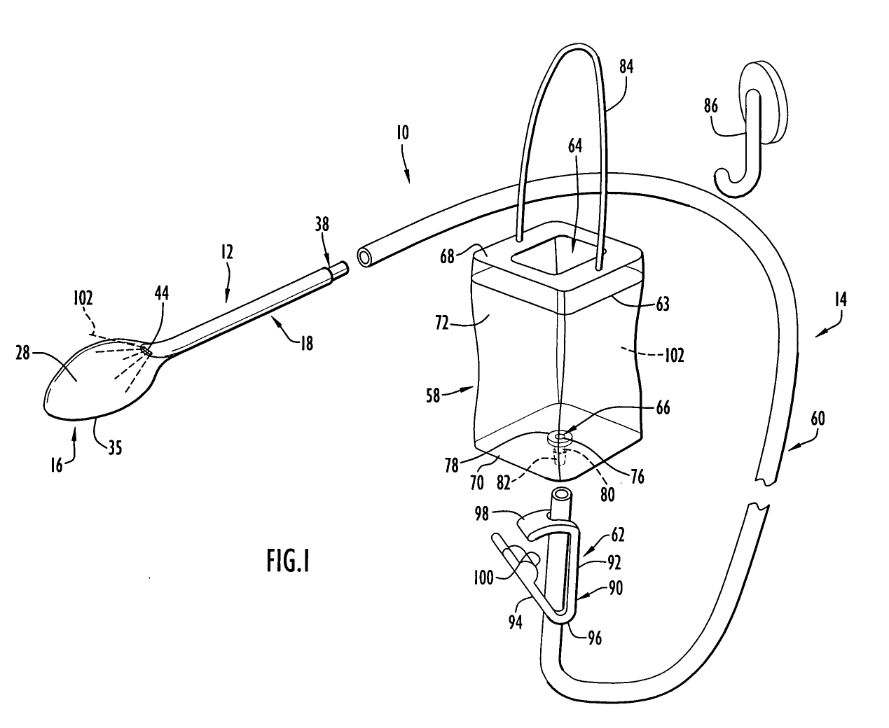

A tongue deodorizing device 10 is depicted in FIG. 1 and comprises a tongue contacting instrument 12 and a water or fluid supply 14 removably connectable to the tongue contacting instrument 12. The tongue contacting instrument 12, as best shown in FIGS. 1-6, comprises a tongue contacting member 16 and a handle 18 connected to the tongue contacting member 16. The tongue contacting member 16 has a distal or forward end 20 and has a proximal or rearward end 22 spaced longitudinally from the forward end 20. The forward end 20 defines a distal or forward end of the tongue contacting instrument 12. The rearward end 22 is connected with the handle 18. The tongue contacting member 16 is bisected lengthwise or longitudinally by a longitudinal plane P1 containing a central longitudinal axis X of the handle 18. The central longitudinal axis X is also the central longitudinal axis of the tongue contacting instrument 12, and the entire instrument 12 is bisected longitudinally by the longitudinal plane P1. The tongue contacting member 16 has a length or longitudinal dimension in the longitudinal plane P1 and has a width or lateral dimension perpendicular to its length and to the longitudinal plane P1. The tongue contacting member 16 comprises at least one tongue contacting side or face 24 and, preferably, comprises opposed tongue contacting sides or faces 24 and 26. The tongue contacting side 24 may be considered a first tongue contacting side and comprises a stainless steel tongue rubbing surface 28 for being rubbed against the surface of the tongue. The tongue contacting side 26 may be considered a second tongue contacting side and comprises a tongue scraping element 30 for scraping the surface of the tongue and a debris collecting surface 32 bordered by the tongue scraping element 30 for collecting debris scraped from the surface of the tongue by the tongue scraping element 30. The tongue scraping element 30 extends continuously and without interruption from one lateral side of rearward end 22 to the opposite lateral side of rearward end 22.

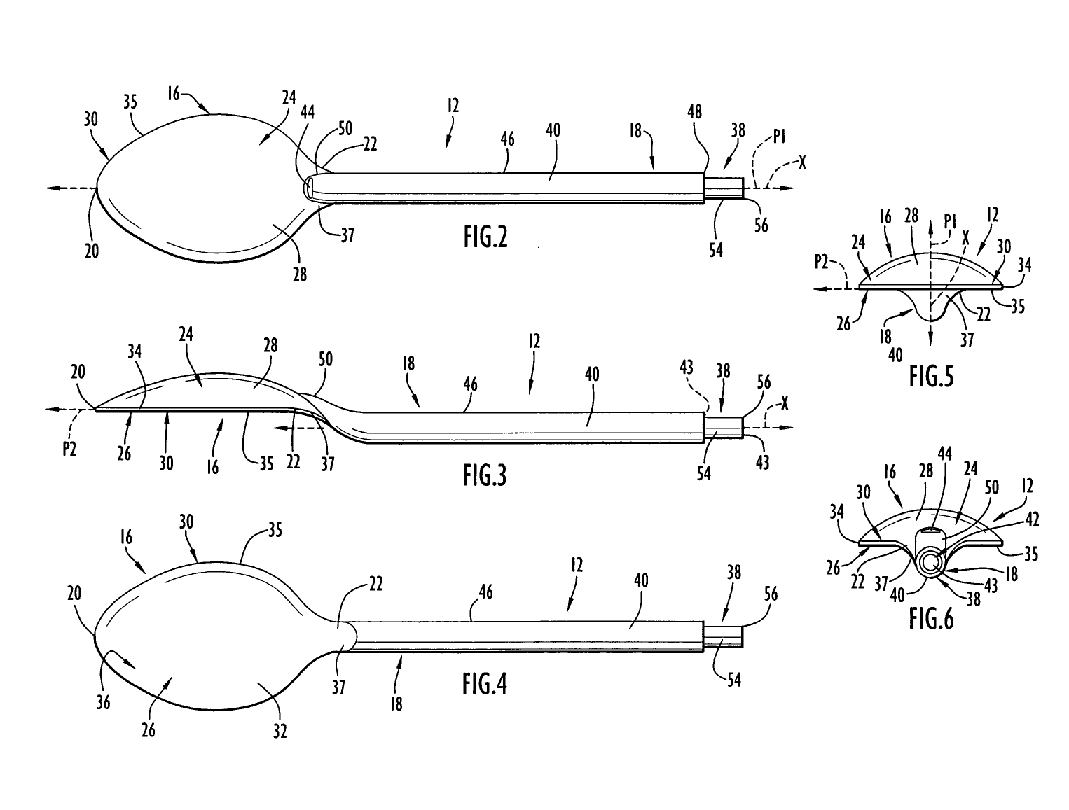

The tongue rubbing surface 28 is a smooth, solid surface bordered or bounded by the tongue scraping element 30. The tongue rubbing surface 28 extends in the longitudinal direction between the forward and rearward ends 20 and 22, and extends in the lateral direction perpendicular to the longitudinal direction. The tongue rubbing surface 28 is preferably continuously curving with a convex curvature in the lengthwise or longitudinal direction, and with a convex curvature in the widthwise or lateral direction. The configuration of the tongue rubbing surface 28 may be considered similar to the exterior surface configuration of a dome or bowl. As depicted in FIG. 3, the longitudinal convex curvature of the tongue rubbing surface 28 may be somewhat asymmetrical, having a steeper or more pronounced convex curvature extending distally from rearward end 22 and having a more gradual or less steep convex curvature extending proximally from distal end 20. The tongue rubbing surface 28 may thusly be composed of surface segments of different radii of curvature. For example, the longitudinal convex curvature of the tongue rubbing surface 28 may have a proximal radius of curvature extending distally from rearward end 22 and may have a distal radius of curvature, greater than the proximal radius of curvature, extending proximally from distal end 20. Thus, the elevation of the tongue rubbing surface 28 from the tongue scraping element 30 in the longitudinal direction may increase more gradually in the proximal direction from distal end 20 than the elevational increase in the distal direction from rearward end 22. It should be appreciated, however, that the tongue rubbing surface 28 can have a symmetrical longitudinal convex curvature. The lateral convex curvature of the tongue rubbing surface 28 may be symmetrical on each side of plane P1 as shown in FIGS. 5 and 6. The configuration of the tongue rubbing surface 28 is advantageous for establishing close contact with the surface of the tongue. When the tongue rubbing surface 28 is pressed against and rubbed along the surface of the tongue, the tongue will deformably conform or substantially deformably conform to the configuration of the tongue rubbing surface to establish close contact with at least a substantial portion of the tongue rubbing surface.

The tongue scraping element 30 extends along the perimeter of the tongue contacting member 16 and is preferably continuously curving between the lateral sides of rearward end 22. The tongue scraping element 30 may follow a substantially elliptical, ovoid or oblong configuration or path between the lateral sides of rearward end 22. Accordingly, the length of the tongue contacting member 16 along the longitudinal plane P1 may be greater than the maximum width of the tongue contacting member 16 perpendicular to the longitudinal plane P1. The forward end 20 of the tongue contacting member 16 is located along the tongue scraping element 30 in alignment with the longitudinal plane P1. The tongue scraping element 30 comprises one or more tongue scraping edges and/or tongue scraping surfaces for scraping the surface of the tongue. The tongue scraping element 30 comprises a tongue scraping surface 34 of narrow width connecting the tongue rubbing surface 28 to the debris collecting surface 32, and a tongue scraping edge 35 formed by the juncture of tongue scraping surface 34 with the debris collecting surface 32. The tongue scraping edge 35 is disposed in a lateral plane P2, perpendicular to the longitudinal plane P1, except for rearward end segments of the tongue scraping edge 35 which curve angularly from the lateral plane P2. As best depicted in FIGS. 3 and 5, the lateral plane P2 containing the tongue scraping edge 35 is offset from the central longitudinal axis X in parallel relation therewith. The direction in which the tongue rubbing surface 28 is elevated from the lateral plane P2 and from the tongue scraping element 30 is perpendicular to the lateral plane P2. Both the tongue scraping surface 34 and the tongue scraping edge 35 can be used to scrape the surface of the tongue to remove material therefrom, but the angled corner formed at edge 35 will be most advantageous for removing adherent material from the surface of the tongue.

The debris collecting surface 32 is a smooth, solid surface bounded or bordered by the tongue scraping edge 35. The debris collecting surface 32 preferably has a concave curvature that follows the convex curvature of the tongue rubbing surface 28. Accordingly, the debris collecting surface 32 may have a longitudinal concave curvature that follows the longitudinal convex curvature of the tongue rubbing surface 28, and the debris collecting surface 32 may have a lateral concave curvature that follows the lateral convex curvature of the tongue rubbing surface 28. The configuration of the debris collecting surface 32 may thusly be considered similar to the interior surface of a dome or bowl. The debris collecting surface 32 defines a debris collecting cavity, depression or recess 36 bordered or bounded by the tongue scraping edge 35.

The rearward end 22 of the tongue contacting member 16 comprises a neck 37 that tapers proximally in width between its lateral sides to connect with handle 18. The neck 37 also curves angularly from the lateral plane P2 to connect with the handle 18, which establishes the parallel offset between the lateral plane P2 of the tongue scraping edge 35 and the central longitudinal axis X of the handle 18. The configuration of the neck 37 provides a smooth tapered transition between the tongue contacting member 16 and the handle 18. The tongue scraping element 30, including the tongue scraping surface 34 and the tongue scraping edge 35, may extend along the neck 37 but these portions or segments of the tongue scraping element 30 will not generally be used to scrape the tongue. Rather, the portion of the tongue scraping element 30 that lies in or along the lateral plane P2 will in general be used to scrape the tongue.

The handle 18 includes a handle body having a distal or forward end joined to the rearward end 22 or neck 37 of the tongue contacting member 16 and having a rearward or proximal end 38 defining a rearward or proximal end of the tongue contacting instrument 12. A water or fluid transport channel 42 extends within the handle body and has a water or fluid inlet 43 at the proximal end 38 and has a water or fluid outlet 44 adjacent the tongue rubbing surface 28. The proximal end 38 of the handle 18 is removably connectible to the water supply 14 to supply water to the fluid inlet 43. The handle 18 has a handle body comprising a longitudinally elongate outer member 40, the water or fluid transport channel 42 extending along the outer member 40, the water or fluid inlet 43 at the proximal end 38 in communication with the fluid transport channel for receiving water from the water supply 14, and the water or fluid outlet 44 adjacent the tongue rubbing surface 28 for discharging water from the fluid transport channel 42. The outer member 40 has a cylindrical portion 46 coaxial with the central longitudinal axis X and extending distally from a terminal proximal end 48 of the cylindrical portion 46 to an angled or sloping distal extension 50 of the outer member 40. The distal extension 50 extends distally from the cylindrical portion 46 at an angle in close overlapping relation over the rearward end 22 or neck 37 of the tongue contacting member 16 to terminate at a terminal distal end 52 of the outer member 40. The terminal distal end 52 can be rounded, arcuate or blunt and can have a convex curvature. The distal extension 50 can be angled and/or curved to follow or conform to the angle and/or curve of rearward end 22 or neck 37, which extends distally or forwardly from the handle at an angle to the central longitudinal axis X. The fluid outlet 44 is located in the terminal distal end 52 and is disposed over and adjacent the tongue rubbing surface 28 at a location adjacent, near or close to the rearward end 22 or neck 37 such that water discharged from the fluid outlet 44 is caused to flow over at least a substantial portion of the tongue rubbing surface 28. The fluid outlet 44 is preferably centered with respect to the width of the tongue contacting member 16 and, therefore, the fluid outlet 44 can be in longitudinal alignment with the distal end 20 of the tongue contacting member 16 along the longitudinal plane P1. The fluid outlet 44 comprises one or more apertures, slots or holes of any desirable configuration in the terminal distal end 52. The fluid outlet 44 is depicted by way of example as an elongate, slightly curved slot extending in a direction transverse to the longitudinal plane P1.

The fluid outlet 44 is in fluid communication with the fluid transport channel 42, and the fluid transport channel 42 can extend within the outer member 40. Accordingly, the terminal proximal end 48 of the outer member 40 can be an open end serving as the fluid inlet 43 as shown in dotted lines in FIG. 3, and the outer member 40 can be tubular or at least partly hollow to contain the fluid transport channel 42 therein in communication with the open terminal proximal end 48 and with the fluid outlet 44. The open terminal proximal end 48 of outer member 40 can be receivable with a snug friction fit within an end of a conduit of water supply 14 to supply water to the fluid transport channel for discharge from the fluid outlet 44 as explained further below.

It is also possible to design the handle 18 with a handle body that includes an inner member 54 disposed within the outer member 40 as depicted in FIGS. 1-4 and 6. The inner member 54 can extend proximally from the open terminal proximal end 48 of the outer member 40 to an inner member terminal proximal end 56. The proximal extension of the inner member 54 beyond the terminal proximal end 48 of the outer member 40 can be receivable with a snug friction fit within an end of the conduit of water supply 14 to supply water to the fluid transport channel for discharge from the fluid outlet 44 and assist removable connection of the proximal end of the handle 18 to the water supply 14 as discussed further below. The terminal proximal end 56 of the inner member 54 can be an open end serving as the fluid inlet 43, as shown in solid lines in FIG. 3, and the inner member 54 can be tubular or at least partly hollow to contain the fluid transport channel therein in communication with the open terminal proximal end 56 and with the fluid outlet 44. Alternatively, the inner member 54 can have an external or outer dimension sufficiently smaller than the internal or inner dimension of the outer member 40 so that the fluid transport channel 42 is defined by a gap or space between the internal surface of the outer member 40 and the external surface of the inner member 54, which gap or space is placed in communication with the conduit of water supply 14 by reception of the terminal proximal end 48 within an end of the conduit. It should be appreciated that any suitable arrangement providing fluid communication between a fluid inlet at the proximal end of the handle 18 and the fluid outlet 44 in the terminal distal end 52 of the handle can be used as the fluid transport channel 42.

In a representative but not limiting example of a tongue contacting instrument 12, the tongue contacting instrument has an overall length from distal end 20 to the terminal proximal end 56 of or about 6.5 inches and has a maximum width, corresponding to the maximum width of the tongue contacting member 16, of or about 1.31 inches. At least the tongue rubbing surface 28 is made of stainless steel, and it is desirable that at least the tongue contacting member 16 be made in its entirety of stainless steel. Preferably, the entire tongue contacting instrument 12 is made of stainless steel. The tongue contacting member 16 and the handle 18 can be made integrally, unitarily or monolithically as a single part. The tongue contacting member 16 can be made separately from the handle 18 and assembled to the handle in any suitable manner. Where the handle 18 includes an inner member within the outer member, the outer and inner members can be separate components assembled in any suitable manner.

The water supply 14 is depicted in FIG. 1 and comprises a container 58 for holding a quantity of water, a conduit 60 for connecting the container 58 to the fluid inlet 43 of the handle 18, and a flow control mechanism 62 for controlling the flow of water from container 58 to the fluid inlet 43. The container 58 can have any suitable configuration and, preferably, the container 58 has a configuration and size to hold about 6-8 ounces of water. The container 58 can include indicia 63, such as a fill line, indicating the level to which the container must be filled with water to obtain a quantity of water within the container in the range of about 6-8 ounces. The container 58 can be made of transparent material or can be provided with a transparent window allowing the water level in the container to be visualized for alignment of the water level with the indicia 63. A supply orifice or port 64 is provided in a top of the container 58 for supplying plain water to the container from an available water source such as a tap or faucet. A discharge orifice or port 66 is provided in a bottom of the container 58 for discharging or releasing water from the container via gravitational force.

The container 58 is depicted by way of example as comprising a top wall 68, a bottom wall 70 and four side walls 72 connecting the top and bottom walls. The top wall 68 and the bottom wall 70 may have a square peripheral configuration with rounded corners, and the side walls 72 can be arranged to form or meet at rounded corners corresponding to those of the top and bottom walls. The supply orifice 64 includes an aperture in the top wall 68 of any suitable peripheral configuration. The supply orifice 64 may be centered in the top wall 68. The indicia 63 is depicted as a fill line on the side walls 72 extending around the entire periphery of the container. The discharge orifice 66 is formed by a passage extending entirely through a coupling member 76 located in the bottom wall 70. The coupling member 76 includes a sealing disk 78 disposed on or in the bottom wall 70 along the interior side of the container 58 and a stem 80 extending from the sealing disk 78 through the bottom wall 70 to a connector element 82 located exteriorly of the container 58. The discharge orifice 66 extends through the sealing disk 78, the stem 80 and the connector element 82 in a vertical direction and may be centrally located in the bottom wall 70. The connector element 82 can have a truncated conical configuration or any suitable configuration to be removably received within an end of conduit 60 with a snug friction fit. The container 58 can be provided with a handle 84 by which the container can be suspended or hung from an available structure, such as a door knob 86, at an elevation to assist the gravitational discharge of water from the container through the coupling member 76. The handle 84 can have any desirable configuration and is illustrated as a bail having ends attached to the top wall 68. The container 58 can be made of various materials including rigid materials and/or resilient, flexible or deformable materials. Preferably, the configuration of the container and the materials from which it is made facilitate thorough cleaning for reuse. In a representative but not limiting example, the container 58 has a height of or about 3¼ inches, a width of or about 1¾ inches, and a depth of or about 1¾ inches.

The conduit 60 comprises a length of flexible tubing or hose having open ends removably connectible with the proximal end of handle 18 and with the connector element 82 of coupling member 76 and having a lumen extending therethrough. The conduit 60 is of sufficient length to extend from the container 58 to the instrument 12 while allowing the tongue contacting member 16 to be placed in the mouth of the user. It is preferred that the conduit 60 have a tubular wall that is resiliently collapsible or deformable.

The flow control mechanism 62 can be designed in various ways to control the flow of water from container 58 into the fluid transport channel 42 of the tongue contacting instrument 12. The flow control mechanism 62 comprises a clamp 90 mounted on the conduit 60 near the end of the conduit that is to be connected with the coupling member 76. The clamp 90 includes a bracket 92 removably attached on the conduit 60, a clamp arm 94 pivotally connected to an end of the bracket 92 by a hinge 96, and a locking arm 98 extending from an end of the bracket 92 opposite the hinge. The bracket 92 is formed with a recess for retentively receiving the conduit 60 in a removable manner. Although it may be preferable for the bracket 92 to be placed on the conduit 60 near the end of the conduit that is to be connected to the coupling member 76, the bracket 92 can be placed at other locations along the conduit 60 by removable retention of the conduit in the recess of the bracket. The clamp arm 94 is angularly connected to the bracket 92 via the hinge 96 and extends toward the locking arm 98. The clamp arm 94 carries a protuberance 100 that faces toward the conduit 60. The clamp arm 94 has a slot or notch, not visible in FIG. 1, for receiving the locking arm 98 in releasable locking engagement when the clamp 90 is in a closed or clamped position.

The clamp 90 is depicted in FIG. 1 in an open or unclamped position in which the locking arm 98 is not received in the slot in the clamp arm 94 and the protuberance 100 does not press against the conduit 60 in a constricting manner. Accordingly, the lumen through the conduit 60 is not constricted in size and fluid flow through the conduit is not restricted. The clamp 90 is moved from the open position to the closed position by manually pivoting the clamp arm 94 toward the conduit 60 in the direction of the arrow in FIG. 1. Pivotal movement of the clamp arm 94 toward the conduit 60 causes the locking arm 98 to enter the slot in the clamp arm 94 and causes the protuberance 100 to press against and deform the conduit 60 in a constricting manner. The locking arm 98 will be in locking engagement with the clamp arm 94 when the clamp 90 is in the closed position, and the locking engagement between the locking arm 98 and the clamp arm 94 can be obtained through a frictional and/or mechanical interengagement. In the case of clamp 90, the locking arm 98 is provided with a curved configuration to assist frictional retention of the locking arm in the slot of clamp arm 94 when the clamp is in the closed position. In the closed position for clamp 90, the protuberance 100 pressing against the conduit 60 collapses the conduit wall and constricts or closes the lumen of the conduit to restrict or prevent fluid flow past the clamp 90. The clamp 90 can be moved from the closed position to the open position by manually releasing the clamp arm 94 from engagement with the locking arm 98. The clamp 90 can be provided with or made from materials providing a spring bias by which the clamp 90 is normally disposed in or assumes the open position.

When the conduit 60 is connected with the fluid inlet 43 of handle 18 and with the coupling member 76 of container 58 and the clamp 90 is in the open position, water is discharged from the container 58 into the conduit 60 due to gravitational force, and the water flows through the conduit 60 and the fluid transport channel 42 of the tongue contacting instrument 12 for discharge from the fluid outlet 44. The tongue deodorizing device 10 is designed so that the water is discharged from the fluid outlet 44 at a flow rate of or about 0.2 ounce-0.4 ounce per second. Accordingly, a quantity of or about 6-8 ounces of water in container 58 is discharged from the fluid outlet 44 in about 20-30 seconds.

The tongue deodorizing device 10 can be used to accomplish or perform two different modes of tongue deodorization. One mode of tongue deodorization involves a mechanical scraping action performed with the tongue scraping element 30 of the tongue contacting instrument 12. A second mode of tongue deodorization involves a non-mechanical reaction that is caused to occur due to contact of the stainless steel tongue rubbing surface 28 with the tongue in the presence of the flow of water 102 discharged from the fluid outlet 44. The mechanical mode of tongue deodorization can be performed with or without a flow of water provided to the mouth. In addition, it is preferable to perform the mechanical mode of tongue deodorization first, closely followed by performance of the non-mechanical mode of tongue deodorization.

In order to perform the mechanical mode of tongue deodorization, the user manually grasps the handle 18 and manipulates the tongue contacting instrument 12 via the handle to place the tongue scraping element 30 in contact with the tongue, and especially the dorsum of the tongue. The user manipulates the handle 18 to press the tongue scraping element 30 against the tongue while moving it along the surface of the tongue in a scraping action to remove material from the surface of the tongue. Typically, one lateral side of the scraping element 30 will be used to scrape the tongue with the opposite lateral side of the scraping element 30 elevated from the tongue as the tongue contacting member 16 is moved in the direction of the elevated lateral side. It is desirable to contact the dorsum of the tongue with the tongue scraping element 30 as far back or posterior as possible on the tongue, within comfort limits of the user, and to draw the tongue scraping element forwardly or anteriorly along the tongue in a posterior to anterior scraping stroke, but scraping strokes can be performed in any direction along the surface of the tongue. Desirably, the scraping strokes will be repeated numerous times to remove as much material as possible from the tongue and, in particular, the coating on the dorsum of the tongue. Either or both of the tongue scraping surface 34 and the tongue scraping edge 35 can be used to scrape material from the tongue. As the tongue scraping element 30 is moved along the surface of the tongue, material scraped from the tongue by the tongue scraping element is directed toward and collected on the debris collecting surface 32 and in the debris collecting cavity 36. Following the mechanical mode of tongue deodorization or at any time during the mechanical mode of tongue deodorization, debris collected on the debris collecting surface 32 and in the debris collecting cavity 36 can be removed by removing the tongue contacting member 16 from the mouth and rinsing the debris collecting surface and cavity with water. Water can be supplied to the mouth during the mechanical mode of tongue deodorization, and water can be supplied to the mouth using the water supply 14 and the fluid transport channel of the tongue contacting instrument 12 as explained below.

In order to perform the non-mechanical mode of tongue deodorization, the ends of conduit 60 are respectively connected to the proximal end 38 of handle 18 and to the connector element 82 of container 58 to place the fluid inlet 43 in fluid communication with the container 58, which is supplied with the designated quantity of water 102. Plain ordinary water is supplied to the container 58 from an available water source, such as a tap or faucet, via the supply orifice 64. At this point in the procedure, the clamp 90 will be in the closed position so that the water does not yet flow into the fluid inlet 43 and fluid transport channel 42 of the tongue contacting instrument 12. The user manually grasps the handle 18 and manipulates the tongue contacting instrument 12 via the handle 18 to place the tongue rubbing surface 28 in contact with the tongue, and especially the dorsum of the tongue. The user manipulates the handle 18 to press the tongue rubbing surface 28 against the tongue and releases the clamp 90 for movement to the open position so that the water 102 flows by gravity from the container 58 and is discharged from the fluid outlet 44 of the tongue contacting instrument 12. While the water 102 is being discharged from the fluid outlet 44, the user manipulates the handle 18 to move the tongue rubbing surface 28 along the surface of the tongue. The tongue rubbing surface 28 is rubbed along as much as possible of the dorsum of the tongue, within comfort limits of the user. The tongue rubbing surface 28 can be rubbed against the surface of the tongue using back and forth rubbing strokes in any direction on the tongue. The water 102 is discharged from the fluid outlet 44 onto the tongue rubbing surface 28, as well as onto the tongue surface being contacted therewith. The water flows longitudinally and laterally over the tongue rubbing surface 28 as well as between the tongue rubbing surface and the surface of the tongue. The stainless steel tongue rubbing surface 28 in contact with the tissue of the tongue is reactive with the water 102 to bring about a non-mechanical reaction that reduces odor arising from the tongue. The quantity and flow rate of water 102 from the fluid outlet 44 ensures that a sufficient amount of water is present in the region where the tongue rubbing surface 28 contacts the surface of the tongue to effectively bring about the deodorizing reaction. The tongue rubbing surface 28 is rubbed against the tongue until the entire quantity of water in container 58 has been discharged from the fluid outlet 44. The first and second modes of tongue deodorization can be performed separately or in combination at desired intervals to deodorize the tongue.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A tongue deodorizing device comprising
a tongue contacting instrument including a tongue contacting member and a handle connected to said tongue contacting member, said tongue contacting member having a forward end and having a rearward end longitudinally spaced from said forward end, said rearward end being connected to said handle, said tongue contacting member having a tongue rubbing surface for being rubbed against the surface of the tongue, said tongue rubbing surface being a smooth, solid stainless steel surface reactive with plain water to reduce odor from the tongue via a non-mechanical reaction, said tongue rubbing surface extending in a longitudinal direction between said forward and rearward ends, said handle including a handle body and a fluid transport channel within said handle body, said handle body including a forward end connected to said rearward end of said tongue contacting member and including a rearward end, said fluid transport channel having a fluid inlet at said rearward end of said handle body and a fluid outlet adjacent said tongue rubbing surface near said rearward end of said tongue contacting member; and
a water supply removably connectible with said fluid inlet for supplying plain water to said fluid transport channel to be discharged from said fluid outlet to react with said tongue rubbing surface, said water supply including a container for holding a quantity of water, a conduit for connecting said container in fluid communication with said fluid inlet, and a flow control mechanism for controlling water flow from said container to said fluid inlet.

2. The tongue deodorizing device recited in claim 1 wherein said handle body has a central longitudinal axis, said tongue contacting member is bisected in said longitudinal direction by a longitudinal plane containing said central longitudinal axis, said tongue rubbing surface extends in a lateral direction perpendicular to said longitudinal direction, said tongue rubbing surface has a convex curvature in said longitudinal direction and has a convex curvature in said lateral direction

3. The tongue deodorizing device recited in claim 2 wherein said handle body includes an elongate outer member having a cylindrical portion coaxial with said central longitudinal axis, said rearward end of said tongue contacting member includes a neck extending forwardly from said cylindrical portion at an angle to said central longitudinal axis, said outer member further includes a distal extension extending forwardly from said cylindrical portion exteriorly along said neck to terminate at a terminal distal end, said fluid outlet being located in said terminal distal end, said cylindrical portion having an open terminal proximal end, said fluid transport channel extending through said outer member from said fluid inlet to said fluid outlet.

4. The tongue deodorizing device recited in claim 3 wherein said handle body further comprises an inner member disposed within said outer member, said inner member extending proximally from said terminal proximal end of said outer member to define a proximal extension for being removably connectably received in an end of said conduit, said inner member having an open terminal proximal end defining said fluid inlet, said fluid transport channel extending through said inner member.

5. The tongue deodorizing device recited in claim 1 wherein said fluid outlet is in longitudinal alignment with said forward end of said tongue contacting member.

6. The tongue deodorizing device recited in claim 5 wherein said tongue contacting member has a perimeter and further includes a tongue scraping edge extending along said perimeter, said forward end of said tongue contacting member being located along said tongue scraping edge.

7. The tongue deodorizing device recited in claim 6 wherein said tongue contacting member further includes a debris collecting surface and said tongue scraping edge is formed by a narrow surface of said tongue contacting member connecting said tongue rubbing surface to said debris collecting surface.

8. The tongue deodorizing device recited in claim 7 wherein said debris collecting surface defines a concave debris collecting cavity bordered by said tongue scraping edge.

9. The tongue deodorizing device recited in claim 1 wherein said container holds a quantity of about 6-8 ounces of water and said fluid outlet discharges the entire quantity of water in about 20-30 seconds.

10. A tongue deodorizing device comprising
a tongue contacting instrument including a tongue contacting member and a handle connected to said tongue contacting member, said tongue contacting member including a first tongue contacting side and a second tongue contacting side opposite said first tongue contacting side, said first tongue contacting side including a tongue rubbing surface for being rubbed against the tongue, said tongue rubbing surface being a smooth, solid stainless steel surface reactive with plain water to reduce odor from the tongue via a non-mechanical reaction, said second tongue contacting side including a tongue scraping edge for scraping the tongue to reduce odor from the tongue via a mechanical action, said handle including a forward end connected to said tongue contacting member, a rearward end, and a fluid transport channel extending through said handle, said fluid transport channel having a fluid inlet at said rearward end of said handle and a fluid outlet adjacent said tongue rubbing surface; and
a water supply removably connectible with said fluid inlet for supplying plain water to said fluid transport channel to be discharged from said fluid outlet to react with said tongue rubbing surface.

11. The tongue deodorizing device recited in claim 10 wherein said handle includes a central longitudinal axis, said tongue contacting instrument is bisected longitudinally by a longitudinal plane containing said central longitudinal axis, said tongue scraping edge is disposed in a lateral plane perpendicular to said longitudinal plane, said lateral plane being offset from said central longitudinal axis in parallel spaced relation.

12. The tongue deodorizing device recited in claim 11 wherein said tongue scraping edge follows a continuously curving path in said lateral plane.

13. The tongue deodorizing device recited in claim 12 wherein said tongue contacting member further includes a tapered neck connecting said tongue contacting member to said forward end of said handle, said neck being angled from said lateral plane.

14. The tongue deodorizing device recited in claim 12 wherein said second tongue contacting side further includes a concave debris collecting surface bordered by said tongue scraping edge.

15. The tongue deodorizing device recited in claim 14 wherein said tongue contacting member is made in its entirety of stainless steel.

16. The tongue deodorizing device recited in claim 14 wherein said tongue contacting instrument is made in its entirety of stainless steel.

17. The tongue deodorizing device recited in claim 10 wherein said water supply includes a container for holding a quantity of water, a flexible conduit removably connectible with said container and with said tongue contacting instrument for delivering water from said container to said fluid inlet, and a flow control mechanism for controlling the flow of water to said fluid inlet, said container including a coupling member through which water is released from said container by gravity, said conduit having a first end removably connectible with said coupling member, a second end removably connectible with said rearward end of said handle, and a lumen extending entirely therethrough, said flow control mechanism including a clamp disposed on said conduit and having an open position wherein said clamp does not restrict the flow of water through said lumen and a closed position wherein said clamp restricts the flow of water through said lumen.

18. A tongue deodorizing device comprising
a tongue contacting instrument including a tongue contacting member and a handle connected to said tongue contacting member, said tongue contacting member having a forward end and having a rearward end longitudinally spaced from said forward end, said rearward end being connected to said handle, said tongue contacting member including a first tongue contacting side and a second tongue contacting side opposite said first tongue contacting side, said first tongue contacting side including a tongue rubbing surface for being rubbed against the tongue, said tongue rubbing surface being a smooth, solid stainless steel surface reactive with plain water to reduce odor from the tongue via a non-mechanical reaction, said tongue rubbing surface extending in a longitudinal direction between said forward and rearward ends and extending in a lateral direction transverse to said longitudinal direction, said tongue rubbing surface having a convex curvature in said longitudinal direction and having a convex curvature in said lateral direction, said second tongue contacting surface including a tongue scraping element for scraping the tongue to reduce odor from the tongue via a mechanical scraping action and a debris collecting cavity for collecting material scraped from the tongue by said tongue scraping element, said handle including a forward end connected to said rearward end of said tongue contacting member, a rearward end and a fluid transport channel extending therethrough, said fluid transport channel having a fluid inlet at said rearward end of said handle and a fluid outlet adjacent said tongue rubbing surface near said rearward end of said tongue contacting member; and
a water supply removably connectible with said fluid inlet for supplying plain water to said fluid transport channel to be discharged from said fluid outlet to react with said tongue rubbing surface, said water supply including a container for holding a quantity of water, a flexible conduit removably connectible with said container and with said tongue contacting instrument for delivering water from said container to said fluid inlet, and a flow control mechanism for controlling the flow of water to said fluid inlet, said container having a bottom and a discharge port along said bottom through which water is discharged from said container by gravity, said conduit having a first end removably connectible with said discharge port, a second end removably connectible with said rearward end of said handle, and a lumen extending entirely therethrough, said flow control mechanism including a clamp disposed on said conduit for selectively restricting the flow of water through said lumen.

19. The tongue deodorizing device recited in claim 18 wherein said debris collecting cavity is formed by a concave debris collecting surface, said tongue scraping element includes a tongue scraping surface connecting said tongue rubbing surface to said debris collecting surface and a tongue scraping edge formed by the junction of said tongue scraping surface with said debris collecting surface.

20. The tongue deodorizing device recited in claim 18 wherein said container includes a supply port at a top of said container for supplying water to the container, indicia on said container indicating the level to which said container should be supplied with water, and a handle for hanging said container from an available structure.
